(19) United States
(12) Patent Application Publication
(10) Pub. No.: US 2025/0252464 A1
(43) Pub. Date: Aug. 7, 2025

(54) COFFEE FLAVOR EVALUATION SYSTEM AND PROCESS

(71) Applicant: Cafe Imports, Inc., Minneapolis, MN (US)

(72) Inventors: Ian Fretheim, Bloomington, MN (US); Jason Long, North Oaks, MN (US)

(21) Appl. No.: 18/981,427

(22) Filed: Dec. 13, 2024

Related U.S. Application Data
(60) Provisional application No. 63/609,745, filed on Dec. 13, 2023.

(51) Int. Cl. G06Q 30/0282

(52) U.S. Cl. CPC G06Q 30/0282

(57) ABSTRACT

A coffee evaluation system and method, the coffee evaluation system and method comprising providing a user interface having a tiered structure of attribute descriptors; and providing an indication of intensity for at least some of the attribute descriptors is disclosed. In an embodiment the coffee evaluation system includes a scoring engine that assigns values to individual entries on the user interface, calculates values for descriptor strings, sorts and tallies category and then sample-level scores, processes the descriptor strings into natural language forms, and selects from a coffee's generated descriptor pool for descriptor output.

This application claims the benefit of U.S. Provisional Application No. 63/609,745, filed Dec. 13, 2023, the content of which is herein incorporated by reference in its entirety.

### FIELD

Embodiments herein relate to systems and processes for evaluation of coffee flavors.

### BACKGROUND

Cupping is a process by which coffee is evaluated. This is how coffee is tasted by producers and buyers around the world to check the quality of a batch of coffee. In cupping, coffees are scored for aspects such as cleanness, sweetness, acidity, flavor, aroma, mouthfeel and aftertaste. According to cupping protocols, hot water is poured onto freshly roasted and ground beans directly into the cup and allowed to steep for 3-5 minutes. The infusion is then mixed and the foamy head removed.

Cupping is a widely used method of analysis for a coffee sample, from its overall quality to individual characteristics (such as acidity or body) and specific flavor notes. At a cupping session, there will typically be a variety of samples. They might be coffee from the same origin but different farms, they could represent different varieties and processing methods, and perhaps they will even be from different countries. This diversity is useful when you are looking to buy or sell coffee, or are simply keen to expand your knowledge and experience.

However, current cupping methods and forms have limitations, and improvements therein are desired.

### SUMMARY

The present disclosure is directed to a system and method for evaluation and characterization of coffee flavor. This system is sometimes referred to as “Coffee Rose”, and is an interactive, dynamic, rich content coffee cupping system utilizing a user independent scoring engine. The cupping form is presented in the format of an electronic flavor wheel where each of the flavors, aromas and tastes displayed are active buttons that can be used to select, combine and endorse applicable coffee descriptors.

The system includes a rich content component that refers to two primary attributes: The first is a tiered structure with the ability to endorse either simple, individual descriptors (such as bitterness, saltiness, acidity, sweetness, sugar browning, herbal, and fruit) or to build and endorse more complex descriptor strings that extend beyond these individual descriptors. The second is an endorsement protocol that uses an indication for intensity for each individual descriptor. These two components allow for a compact and efficient system and method.

The system includes interactive and dynamic components that alter appearance depending on the inputs provided, expanding the active section as well as visually providing reference to the underlying scoring standards without compromising the principle separating the assessor from the final valuation process in favor of focusing them on the descriptive.

In certain embodiments the system and method allow for providing a live indication of connotation, with unused descriptors having a neutral color what matches that of their category (such as the category of a coffee). When selected, descriptors with a net positive connotation are tinted lighter, while those with a net negative connotation are shaded darker. This tinting and shading stacks back down through any prior descriptor strings to the root category button, which displays the net contribution-positive or negative to provide an overall coffee score.

In an example embodiment the coffee rose system is composed of seven attribute categories, four increasingly descriptive tiers, and an intensity indicator. Note that more or fewer attribute categories, descriptive tiers and intensity indicators can be used. These elements combine to create a multi-dimensional, descriptive coffee scoring form that is sensitive not only to the broad qualitative categories that coffee assessors commonly used but also to the degree of specificity noted within each, the specific content of notations, and the quantitative intensity of each impression.

The Coffee Rose is a novel update to coffee cupping methodology as well as to the rapid sensory profiling techniques known as Check All That Apply and Rate All That Apply (CATA and RATA). Where legacy CATA forms allow only for the endorsement of complete or “self-contained” descriptors, the Coffee Rose is structured to allow users to use simple descriptors or combine them to build more complex ones. Where legacy CATA forms lack an indication for intensity, or provide it only at a macro level (for example, indicating that Flavor is strong, but not distinguishing between the strength of multiple possibly present flavors), the Coffee Rose employs a descriptor level intensity indication (such as intense apricot and mild peanut vs intense peanut and mild apricot). Where legacy CATA forms provide descriptors by checking or marking the descriptor on a list, the Coffee Rose introduces a RATA based endorsement protocol where rating the intensity of the descriptor performs the endorsement function. The Coffee Rose provides descriptor input mapping or “locations” such that the relative similarity of descriptors and calibration between assessors can be identified, calculated, and tracked. The Coffee Rose is simultaneously an advanced assessment tool and a digitalized data collection and input tool. The Coffee Rose allows coffee assessors faster, easier, and more experientially aligned inputs. It further normalizes qualitative and subjective inputs for quantitative and objective outputs, and digitalizes data for database storage and processing. The Coffee Rose system and method further digitalizes data for AI applications like machine learning ingestion and language model processing. The Coffee Rose is sensitive to such elements as “category”, “specificity”, “content”, and “intensity”.

For example, with regard to category they provide context for a description but not much descriptive detail. In typical embodiments acidity is measured separately from sweetness.

For example, with regard to specificity, general or specific descriptions can be decoupled from scoring. More specific tier descriptions have greater descriptive power and value impact than more general qualifier tier descriptions.

For example, with regard to content: the specific content of descriptors and descriptor strings are provided. In certain forms content is decoupled from scoring. For example, apricot has greater value in the system than coffee cherry. The difference in value reflects our preference for apricot over coffee cherry. This ensures the descriptors are given equal numeric value for all users, unlike traditional systems.

For example, with regard to intensity, the current system and method is quantitative. Higher intensity descriptors have a greater impact on coffee description and score than lower intensity descriptors.

A scoring engine is typically essential to the system and method. The scoring engine assigns values to individual entries, calculates values for descriptor strings, sorts and tallies category and then sample-level scores, processes the descriptor strings into natural language forms, and selects from a coffee's generated descriptor pool for top-level descriptor output.

Typically attribute scores are generated directly on the basis of descriptive notation and intensity indication. These scores are therefore sensitive both to how attributes are described qualitatively as well as to the quantitative intensities at which those descriptions are observed. While it is still the case that two people can generate different descriptions and arrive at different outcomes for the same coffee, similarity in descriptions reduces differences in scoring when using the system. Further, the differences that arise between cuppers are made computable by the selections and scoring engine, as opposed to when they are individually generated ad hoc.

In an embodiment, the coffee evaluation system includes providing a user interface having a tiered structure of attribute descriptors, and providing an indication of intensity for at least some of the attribute descriptors.

In an embodiment, the coffee evaluation system includes a scoring engine.

In an embodiment, the scoring engine assigns values to individual entries of the attribute descriptors.

In an embodiment, the coffee evaluation system includes attribute descriptor strings of primary attributes and sub-attributes.

In an embodiment, the coffee evaluation system includes attribute descriptor strings of primary attributes, sub-attributes, and sub-sub-attributes.

In an embodiment, the coffee evaluation system includes attribute descriptor strings and a scoring engine that calculates values for descriptor strings.

In an embodiment, the coffee evaluation system includes a scoring engine that sorts and tallies categories and sample-level scores.

In an embodiment, the coffee evaluation system includes a scoring engine that processes descriptor strings into natural language forms.

In an embodiment, the coffee evaluation system includes a scoring engine that selects from a coffee's generated descriptor pool for top-level descriptor output.

In an embodiment, the coffee evaluation system includes a scoring engine that: a) assigns values to individual entries on the user interface, b) calculates values for descriptor strings, c) sorts and tallies category and then sample-level scores, d) processes the descriptor strings into natural language forms, and e) selects from a coffee's generated descriptor pool for descriptor output.

In an embodiment, a first evaluative criteria includes categories that are similar to attributes on a common cupping form and provide context for a description but not descriptive detail.

In an embodiment, specific tier descriptions are provided.

In an embodiment, the system includes specific content descriptors and descriptor strings.

In an embodiment, the descriptor strings are dynamically generated.

In an embodiment, the descriptor strings are dynamically combined.

In an embodiment, the system and method further include evaluation on basis of intensity wherein higher intensity descriptors have a greater impact on coffee description and score than lower intensity descriptors.

In an embodiment, wherein one of the intensity descriptors is a neutral reference.

In an embodiment, the system and methods include four intensity descriptors.

In an embodiment, the system and methods include at least three intensity descriptors.

In an embodiment, the system provides attribute scores that generated directly on the basis of descriptive notation and intensity indication.

In an embodiment, the system includes providing attribute scores sensitive both to how attributes are described qualitatively as well as to a quantitative intensities at which those descriptions are observed.

In an embodiment, the interface is sensitive to the degree of specificity noted within individual categories, the specific content of notations, and the quantitative intensity of each impression.

In an embodiment, the interface is sensitive to the degree of specificity noted within individual categories.

In an embodiment, the interface is sensitive to the degree of specificity noted within specific content of notations.

In an embodiment, the interface is sensitive to the degree of specificity noted with the quantitative intensity of each impression.

In an embodiment, wherein attribute descriptors are composed of seven attribute categories.

In an embodiment, one of the attribute categories includes bitterness.

In an embodiment, one of the attribute categories includes saltiness.

In an embodiment, one of the attribute categories includes acidity.

In an embodiment, one of the attribute categories includes sweetness.

In an embodiment, one of the attribute categories includes sugar browning

In an embodiment, a coffee evaluation system is included having providing a user interface having a tiered structure of attribute descriptors, and providing an indication of intensity for at least some of the attribute descriptors, the system configured to allow users to assigns values to individual entries on the user interface, after which the system: a) calculates values for descriptor strings, b) sorts and tallies category and then sample-level scores, c) processes the descriptor strings into natural language forms, and d) selects from a coffee's generated descriptor pool for descriptor output.

In an embodiment, a first evaluative criteria includes categories that are similar to attributes on a common cupping form and provide context for a description but not descriptive detail.

In an embodiment, the coffee evaluation system includes a scoring engine that sorts and tallies categories and sample-level scores.

In an embodiment, wherein descriptor strings are dynamically combined.

In an embodiment, the system provides attribute scores that generated directly on the basis of descriptive notation and intensity indication.

In an embodiment, the interface is sensitive to the degree of specificity noted within specific content of notations.

In an embodiment, at least three intensity descriptors are included.

In an embodiment, the coffee evaluation system includes a scoring engine that sorts and tallies categories and sample-level scores.

In an embodiment, the interface is sensitive to the degree of specificity noted within individual categories, the specific content of notations, and the quantitative intensity of each impression.

In an embodiment, the scoring engine assigns values to individual entries of the attribute descriptors.

In an embodiment, a coffee evaluation method, the coffee evaluation method is included, the method providing a user interface having a tiered structure of attribute descriptors, and providing an indication of intensity for at least some of the attribute descriptors.

In an embodiment, the coffee evaluation method includes a scoring engine.

In an embodiment, the scoring engine assigns values to individual entries of the attribute descriptors.

In an embodiment, the coffee evaluation method includes attribute descriptor strings of primary attributes and sub-attributes.

In an embodiment, the coffee evaluation method includes attribute descriptor strings of primary attributes, sub-attributes, and sub-sub-attributes.

In an embodiment, the coffee evaluation method includes attribute descriptor strings and a scoring engine that calculates values for descriptor strings.

In an embodiment, the coffee evaluation method includes a scoring engine that sorts and tallies categories and sample-level scores.

In an embodiment, the coffee evaluation method includes a scoring engine that processes descriptor strings into natural language forms.

In an embodiment, the coffee evaluation method includes a scoring engine that selects from a coffee's generated descriptor pool for top-level descriptor output.

In an embodiment, the coffee evaluation method includes a scoring engine that: a) assigns values to individual entries on the user interface, b) calculates values for descriptor strings, c) sorts and tallies category and then sample-level scores, d) processes the descriptor strings into natural language forms, and e) selects from a coffee's generated descriptor pool for descriptor output.

In an embodiment, a first evaluative criteria includes categories that are similar to attributes on a common cupping form and provide context for a description but not descriptive detail.

In an embodiment, wherein specific tier descriptions are provided.

In an embodiment, the method includes specific content descriptors and descriptor strings.

In an embodiment, wherein descriptor strings are dynamically generated.

In an embodiment, wherein descriptor strings are dynamically combined.

In an embodiment, further can include evaluation on basis of intensity wherein higher intensity descriptors have a greater impact on coffee description and score than lower intensity descriptors.

In an embodiment, wherein one of the intensity descriptors is a neutral reference.

In an embodiment, can include four intensity descriptors.

In an embodiment, can include at least three intensity descriptors.

In an embodiment, the method provides attribute scores that generated directly on the basis of descriptive notation and intensity indication.

In an embodiment, the method includes providing attribute scores sensitive both to how attributes are described qualitatively as well as to a quantitative intensities at which those descriptions are observed.

In an embodiment, the interface is sensitive to the degree of specificity noted within individual categories, the specific content of notations, and the quantitative intensity of each impression.

In an embodiment, the interface is sensitive to the degree of specificity noted within individual categories.

In an embodiment, the interface is sensitive to the degree of specificity noted within specific content of notations.

In an embodiment, the interface is sensitive to the degree of specificity noted with the quantitative intensity of each impression.

In an embodiment, wherein attribute descriptors are composed of seven attribute categories.

In an embodiment, wherein one of the attribute categories includes bitterness.

In an embodiment, wherein one of the attribute categories includes saltiness.

In an embodiment, wherein one of the attribute categories includes acidity.

In an embodiment, wherein one of the attribute categories includes sweetness.

In an embodiment, wherein one of the attribute categories includes sugar browning

In an embodiment, wherein one of the attribute categories includes herbal.

In an embodiment, wherein one of the attribute categories includes fruit.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a view of a digital user interface in accordance with various embodiments herein.

FIG. 2 is a view of a digital user interface in accordance with various embodiments herein.

FIG. 3 is a schematic diagram in accordance with various embodiments herein, showing a hierarchy of descriptors and their impact on score.

FIG. 4 is a schematic diagram in accordance with various embodiments herein, showing an example of selected descriptors.

FIG. 5 is a schematic diagram in accordance with various embodiments herein, showing an example of scoring calculations.

FIG. 6 is a schematic diagram in accordance with various embodiments herein, showing intensity selections.

FIG. 7A is view of a digital user interface in accordance with various embodiments herein, showing a series of selections.

FIG. 7B is a view of output from the digital user interface of FIG. 7A.

FIG. 8 shows a basic flowchart of a lightweight cupping tool utilizing the Coffee Rose in accordance with various embodiments herein.

FIG. 9 shows steps for creating a coffee flight, utilizing the Coffee Rose in accordance with various embodiments herein.

FIG. 10 shows an interface for joining a coffee flight, utilizing the Coffee Rose in accordance with various embodiments herein.

FIG. 11 shows an interface for adding a new coffee, utilizing the Coffee Rose in accordance with various embodiments herein.

FIG. 12 shows an interface with an example flight summary, utilizing the Coffee Rose in accordance with various embodiments herein.

FIG. 13 shows an interface with a defect in a coffee analyzed utilizing the Coffee Rose in accordance with various embodiments herein.

FIG. 14 shows an interface that allows inviting other cuppers, utilizing the Coffee Rose in accordance with various embodiments herein.

FIG. 15 shows an example of sharing message in accordance with various embodiments herein.

FIG. 16 shows an example of a mobile application showing the present subject matter in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

The present disclosure is directed to a system and method for evaluation of coffee flavor. This system is sometimes referred to as “Coffee Rose”, and is an interactive, dynamic, rich content coffee cupping system utilizing a user independent scoring engine. The cupping form is presented in the format of a flavor wheel where each of the flavors, aromas and tastes displayed are active buttons that can be used to select, combine and endorse applicable coffee descriptors.

The system includes a rich content component that refers to two primary attribute: The first is its tiered structure with the ability to endorse either simple, individual descriptors or to build and endorse more complex descriptor strings. The second is an endorsement protocol that uses an indication for intensity. These two components allow tor compact and efficient system and method. The system includes interactive and dynamic components that alter appearance depending on the inputs provided, expanding the active section as well as visually providing reference to the underlying scoring standards without compromising the principle separating the assessor from the final valuation process in favor of focusing them on the descriptive.

In certain embodiments the system and method allow for providing a live indication of connotation, with unused descriptors having a neutral color what matches that of their category. When selected, descriptors with a net positive connotation are tinted lighter, while those with a net negative connotation are shaded darker. This tinting and shading stacks back down through any prior descriptor strings to the root category button, which displays the net contribution-positive or negative—that it is making to the overall coffee score.

In an example embodiment the coffee rose system is composed of seven attribute categories, four increasingly descriptive tiers, and an intensity indicator. Note that more or fewer attribute categories, descriptive tiers and intensity indicators can be used. These elements combine to create a multi-dimensional, descriptive coffee scoring form that is sensitive not only to the broad qualitative categories that we are accustomed to but also to the degree of specificity noted within each, the specific content of notations, and the quantitative intensity of each impression.

The Coffee Rose is sensitive to such elements as “category”, “specificity”, “content”, and “intensity”.

For example, with regard to category, similar to attributes on a common cupping form, they provide context for a description but not much descriptive detail. In typical embodiments acidity is measured separately from sweetness.

For example, with regard to specificity, general or specific descriptions can be decoupled from scoring. More specific tier descriptions have greater descriptive power and value impact than more general qualifier tier descriptions.

For example, with regard to content: the specific content of descriptors and descriptor strings are provided. In certain forms content is decoupled from scoring. For example, apricot has greater value in the coffee rose system than coffee cherry. The difference in value reflects our preference for apricot over coffee cherry. This ensures the descriptors are given equal numeric value for all users, unlike traditional systems.

For example, with regard to intensity, the current system and method is quantitative. Higher intensity descriptors have a greater impact on coffee description and score than lower intensity descriptors.

A scoring engine is typically essential to the system and method. The scoring engine assigns values to individual entries, calculates values for descriptor strings, sorts and tallies category and then sample-level scores, processes the descriptor strings into natural language forms, and selects from a coffee's generated descriptor pool for top-level descriptor output.

Typically attribute scores are generated directly on the basis of descriptive notation and intensity indication. These scores are therefore sensitive both to how attributes are described qualitatively as well as to the quantitative intensities at which those descriptions are observed. While it is still obviously the case that two people can generate different descriptions and arrive at different outcomes for the same coffee, similarity in descriptions reduces differences in scoring when using the system. Further, the differences that do arise between cuppers are made computable by the selections and scoring engine, as opposed to when they are individually generated ad hoc.

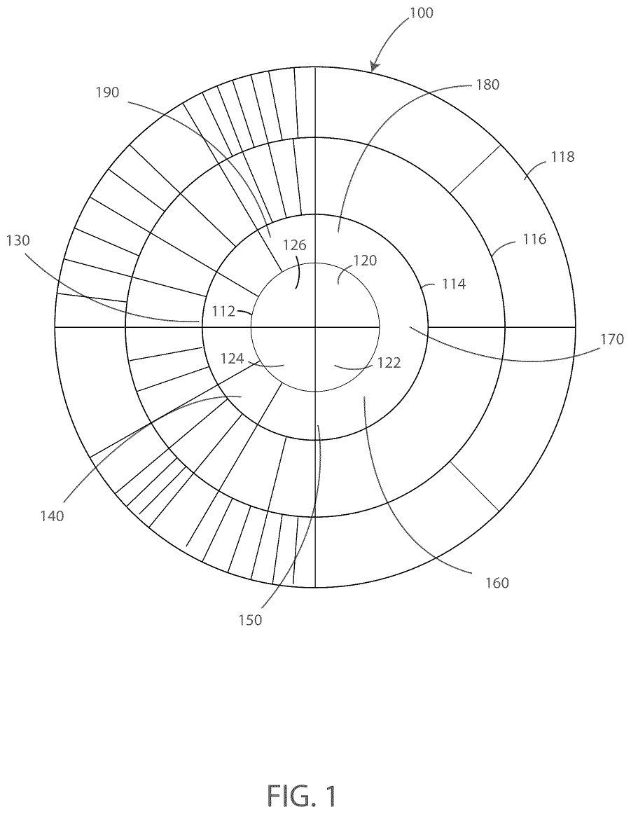

Referring now to the drawings, FIG. 1 is a view of a digital user interface 100 in accordance with various embodiments herein. The digital user interface 100 includes a central intensity selector allowing for the user (the person sampling the coffee) to select from the following levels: reference 120, noticeably 122, significantly 124, and much 126. These intensity selectors are made for each different flavor category. The different flavor categories are herbal 130, sugar browning 140, sweetness 150, acidity 160, saltiness 170, and bitterness 120. Each of these different flavor categories have additional tiers of detail, such as saltiness branch 170 having a first selection tier of 114 for saltiness category, plus a second tier 116 for smooth, and a third tier 118 for soft. The selection of these tiers (such as saltiness, smooth, and soft) combined with an intensity (such as reference, which is low; or much, which is high) will result in a score and descriptor, as described below.

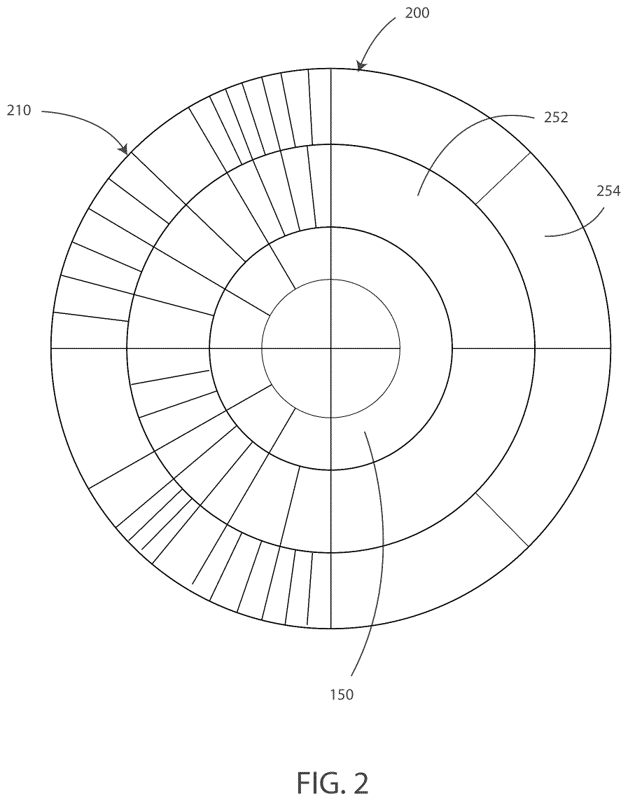

FIG. 2 is a view of a digital user interface 200 in accordance with various embodiments herein. In this view the sweetness flavor category 150 has been selected, resulting in the sweetness category expanding to fill approximately half of the circular user interface selection area. The benefit of this expansion is that it allows for easier viewing of the sweetness category 150 and its tiers. In addition, in some cases there are so many of the top level tiers (see fruit) that they are not easily viewable or selectable) without an expansion. In this case the sweetness category 150 selections include fruit-like 252 and clean 254. While not highlighted as such, one of the intensity criteria at the center would also be selected to indicate how strongly the sweet, fruit-like and clean flavor is detected. Thereafter additional flavor categories can be checked until all impressions of the taster have been recorded. For example, next another sweetness flavor category could be selected (such as sweetness, candy-like, and sugary), or saltiness or fruit or sugar browning . . . could be selected.

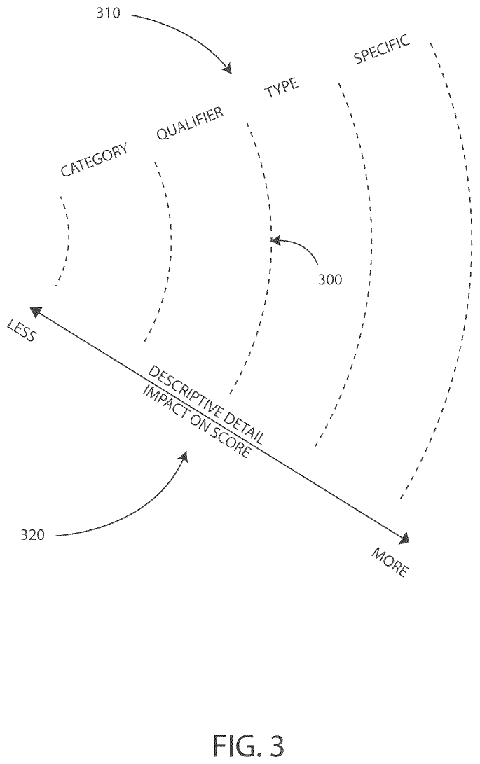

FIG. 3 is a schematic diagram in accordance with various embodiments herein, showing a hierarchy of descriptors and their impact on score. Here we see how the tiers 310 go from overall flavor category to qualifier to type to specific. These tiers become more descriptive as they radiate out to more specificity, and also these tiers have a bigger impact on score as one radiates out, as shown in scale 320

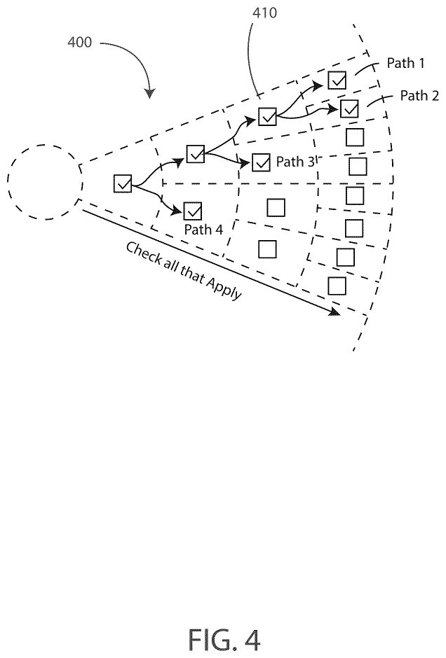

FIG. 4 is a schematic diagram in accordance with various embodiments herein, showing an example of how a group of selections 400 can be made by selecting various descriptors 310. In this example, four different paths are shown: Path 1, Path 2, Path 3, and Path 4. They all have a common first level category, but then diverge at various points.

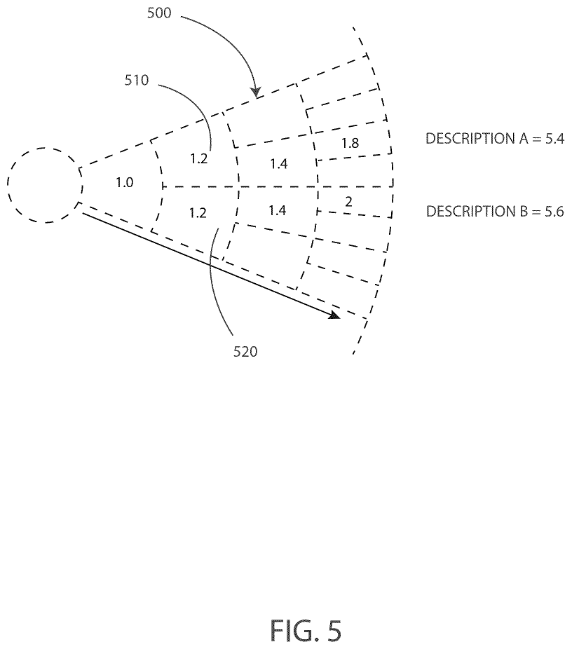

FIG. 5 is a schematic diagram in accordance with various embodiments herein, showing an example of scoring calculations. Here see the overall schematic 500 with two different descriptive paths: Description A with path 510, and Description B with path 520. It will be seen that in this embodiment the totals are added, with each tier having a higher contribution to the score, with there being deviation between the path 510 and 520 at the last tier, where path 510 has a score of 1.8 and path 520 has a score of 2.

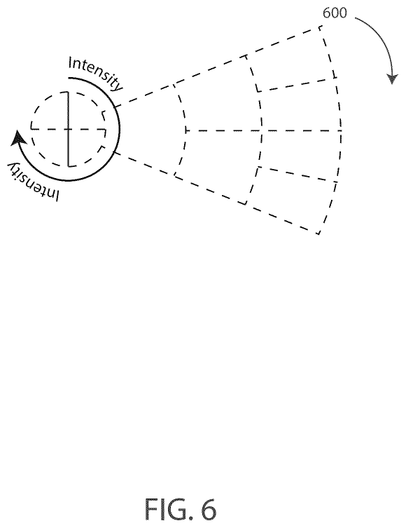

FIG. 6 is a schematic diagram in accordance with various embodiments herein, showing intensity selections interface 600. In this configuration the intensity is higher as one moves clockwise around the selections interface 600. Typically there will be four different intensity selection options, but there can be more or fewer. It is also possible to have a sliding/rotating selector that allows for a wide range of intensities with increased granularity. However, generally there is a benefit in the relative simplicity of the four (or similar) options, which provide predictable and easy options.

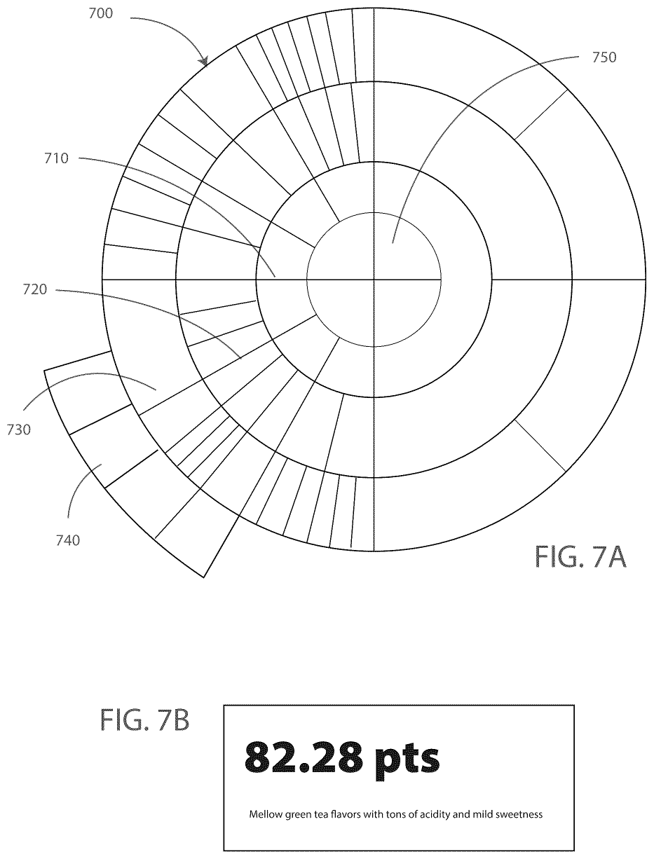

FIG. 7A is view of a digital user interface 700 in accordance with various embodiments herein, showing a series of selections for herbal properties 710, from herbaceous 720 to grassy 730 to green tea 740. The intensity is selected as reference 750, so this is a somewhat mild strength. FIG. 7B is a view of output from the digital user interface 700 of FIG. 7A, showing a score of 82.28 points, with “mellow green tea flavors”. Note that acidity and sweetness were also selected (but not shown expanded), and thus “tons of acidity and mild sweetness”. Is shown.

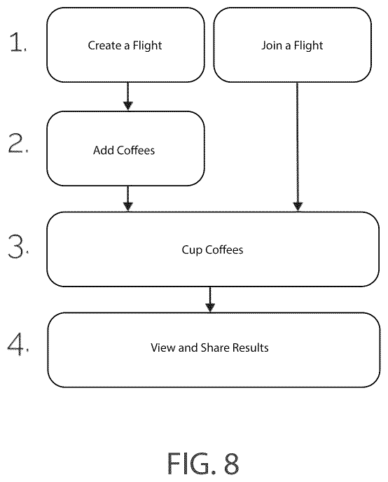

FIG. 8 shows a basic flowchart of a lightweight cupping tool utilizing the Coffee Rose allowing you to quickly build a flight, invite others, cup, and share the results with anyone. It's a web app so can be used on a laptop, desktop or tablet (no mobile support for the moment).

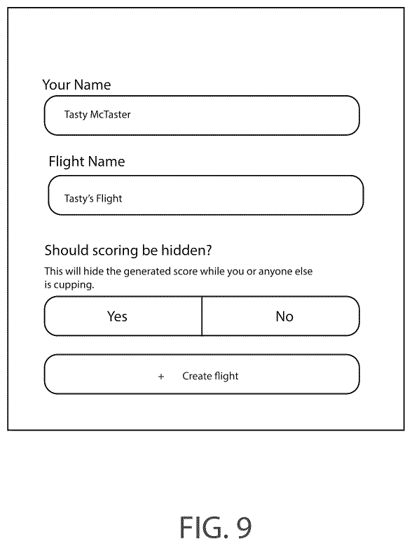

FIG. 9 shows steps for crating a flight, including:
#1. Create a Flight
1. Open DemoX by tapping the Icon on the tablet or visiting the URL:

https://rose-demo.cafeimports.com/x
2. Choose *Create a Flight*
3. Enter your name (this is used to identify each cupper's contribution)
4. Enter a *Flight Name* (this can be anything but usually what the cupping is for). The Coffee Rose will add a date to the flight automatically.
5. Choose if you would like scoring to be hidden while cupping. If you choose *Yes* scoring will be hidden while cupping, but still appear in the Flight Summary.

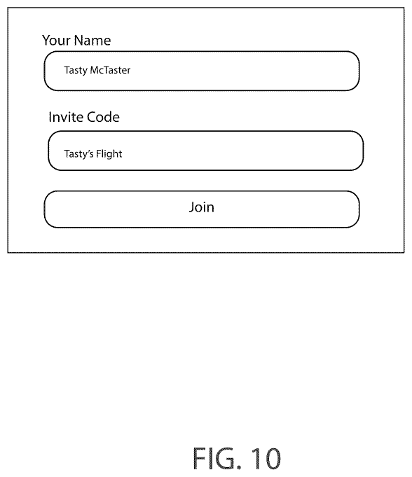

FIG. 10 shows an interface for joining a flight.
1. Open DemoX by tapping the Icon on the tablet or visiting the URL: https://rose-demo.cafeimports.com/x
2. Choose *Join a Flight*
3. Enter your name (this is used to identify your contribution to the flight)
4. Enter the *Invite Code* provided to you by the flight master
5. Choose *Join*

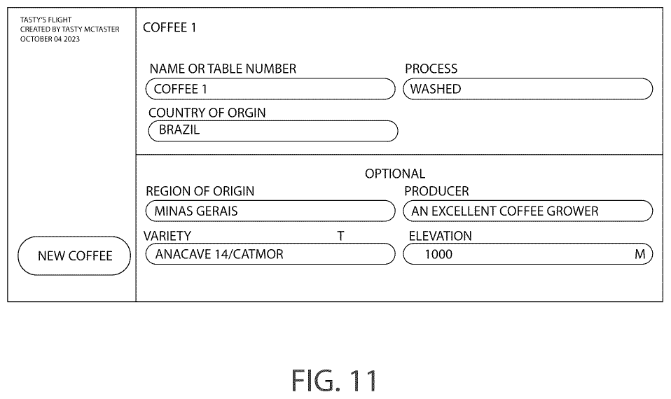

FIG. 11 shows an interface for adding a new coffee.

Your flight will be created but we still need to add the coffees we will be cupping. Some fields are required for identifying the coffee on the table and calculating the final output, while others are optional and for your own reference.

We read from left to right and when skimming an article or image for important things, we generally also start from the left and work to the right. DemoX works the same way. Broad information on the left, and important details or things that need extra attention on the right.

When building your flight or cupping, coffees are listed on the left, and choosing one of them will open their details to the right.

### ** Required **

1. Add a *Name or Table Number* for the coffee.
2. Choose the process the coffee underwent from the *Process* dropdown.
3. Choose the *Country of Origin* from the dropdown.

### ** Optional**

1. Region of Origin
2. Producer
3. Variety
4. Elevation (m/ft)

Once you are done entering the required information and any optional information, choose *Save.*

Note that coffee you just entered changes status from *Incomplete* to *Ready* in the left hand column.

Need to add another coffee? Choose *Add Coffee* in the left hand column to repeat the process above for as many times as needed. DemoX currently supports single extractions of each coffee *.* If you wish to cup multiple extractions of the same coffee, you may do so by adding them as if they were separate coffees to compare the results across cups, however they will not be calculated or displayed collectively in the *Flight Summary*.

Notice that the coffees have a status indicator and color depending on if the coffee is Incomplete (missing required information) Ready (has not been cupped), Complete (has been cupped) or Defective (contains defects).

You can review your work by tapping or clicking the coffee in the left column to see it's details, if you've added all your coffees, choose *Start Cupping* to well . . . start cupping!

### ##Entering Descriptors on the Rose

The Rose allows you to enter what you are experiencing, and the intensity of that experience. It also lets you add information about any defects, and builds a description of the coffee for you as you work.
1. Start by choosing a category (the inner-most ring of the Rose)
2. If you want to add more detail, work your way out each tier of descriptors as the Rose expands. This does not add each one of these items to the description, instead you are adding more detail to that particular category.
   Can't find what you are looking for? Each category allows you to enter anything as an *Other* at the outermost ring.
3. Then choose an intensity from the middle of the Rose from *Reference* (least) to *Much* (most) intense.
4. The Rose resets, ready for the next entry and a checkmark appears next to categories that have at least one descriptor.
   Repeat the above as many times as you like for as many categories or as few categories as it takes to describe the coffee. Experiencing hops and green tea? Add both! The amount of detail you choose to add is completely up to you.

Once you have completed describing all the coffees on the flight, choose

### *** Submit Flight**

##Modifying Entries: If you make a mistake while you are describing, you can *Undo*, *Redo*, or view your complete *History* by choosing the appropriate menu item underneath the description.

###Undo: Undo the last entry.

###Redo: Redo the last action.

###History: The *History* panel shows the descriptors you have entered, the *Intensity* at which you entered them and their effect on the overall score and description as either *Positive* or *Negative.* From this panel you can also remove a single descriptor or remove everything and start over.

###Defects: The Defects panel allows you to enter the number of defects detected in the coffee. Notice that the coffees status changes to Defective to indicate that defects are present.

###The Description: In the automatically generated description, positive descriptors are highlighted green, while negative descriptors are highlighted red. See something inaccurate? Remove it by tapping or clicking on the descriptor.

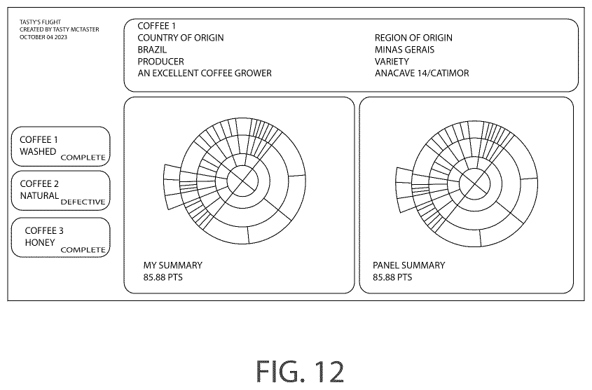

FIG. 12 shows an example flight summary. The flight summary shows your results and a composite result from the data provided by the panel (everyone invited to the flight).

1. Select a coffee from the left column

2. Details about that coffee are shown in the detail area along with your descriptors, the calculated score, the auto-generated description, and any defects that you indicated.

3. The same information is shown in the details area for the panel. This is the composite results for the selected coffee, calculated from the entire panels descriptors.

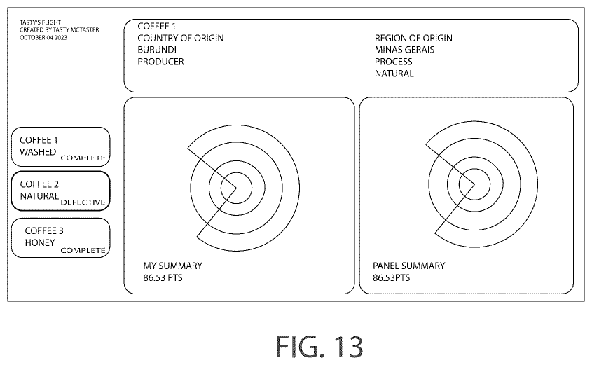

FIG. 13 shows a defect.

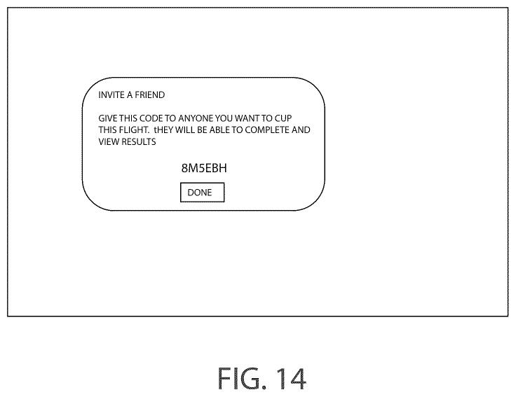

FIG. 14 allows inviting other cuppers.

### ##Invite Other Cuppers to a Flight

1. Choose *Invite to Flight* in the upper right hand corner
2. Send or tell your fellow cuppers the *Invite Code*

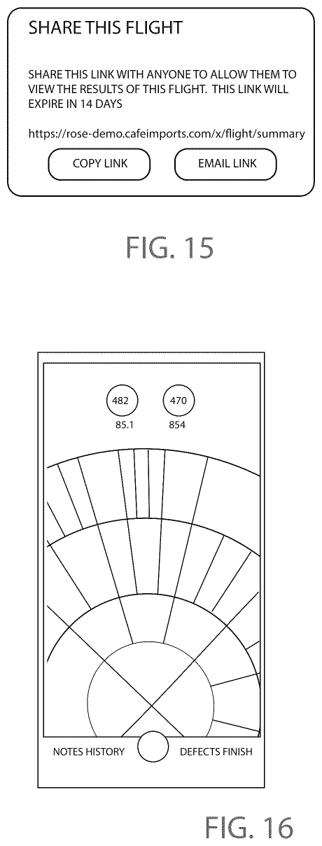

FIG. 15 allows for sharing a flight.

### ##Share Flight Results

Your results are available to share for 14 days from the time they are generated. To share the results:

1. Choose *Share* in the upper right corner.

2. Choose to *Copy Link* for pasting into a message or email.

3. Or choose *Email* to open your email client and automatically fill with the share link.

FIG. 16 shows an example of a mobile application showing the present subject matter.

Once you are finished, you can join another flight, or start over and create your own using the buttons in the lower left.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1.-83. (canceled)

84. A coffee evaluation system, the coffee evaluation system comprising:
providing a user interface having a tiered structure of attribute descriptors; and
providing an indication of intensity for at least some of the attribute descriptors.

85. The coffee evaluation system of claim 84, wherein the coffee evaluation system includes a scoring engine.

86. The coffee evaluation system of claim 84, wherein the coffee evaluation system includes attribute descriptor strings of primary attributes, sub-attributes, and sub-sub-attributes.

87. The coffee evaluation system of claim 84, wherein the coffee evaluation system includes attribute descriptor strings and a scoring engine that calculates values for descriptor strings.

88. The coffee evaluation system of claim 87, wherein each descriptor string has a value.

89. The coffee evaluation system of claim 84, wherein the coffee evaluation system includes a scoring engine that sorts and tallies categories and sample-level scores.

90. The coffee evaluation system of claim 84, wherein the coffee evaluation system includes a scoring engine that selects from a coffee's generated descriptor pool for top-level descriptor output.

91. The coffee evaluation system of claim 84, wherein the coffee evaluation system includes a scoring engine that:
a) assigns values to individual entries on the user interface,
b) calculates values for descriptor strings,
c) sorts and tallies category and then sample-level scores,
d) processes the descriptor strings into natural language forms, and
e) selects from a coffee's generated descriptor pool for descriptor output.

92. The coffee evaluation system of claim 84, further comprising evaluation on basis of intensity wherein higher intensity descriptors have a greater impact on coffee description and score than lower intensity descriptors.

93. The coffee evaluation system of claim 84, wherein the system includes providing attribute scores sensitive both to how attributes are described qualitatively as well as to a quantitative intensity at which those descriptions are observed.

94. The coffee evaluation system of claim 84, wherein the interface is sensitive to the degree of specificity noted within individual categories, the specific content of notations, and the quantitative intensity of each impression.

95. The coffee evaluation system of claim 84, wherein each coffee has a quantitative impression.

96. A coffee evaluation system, the coffee evaluation system comprising:
providing a user interface having a tiered structure of attribute descriptors; and
providing an indication of intensity for at least some of the attribute descriptors; the system configured to allow users to assign values to individual entries on the user interface, after which the system:
a) calculates values for descriptors,
b) sorts and tallies category and then sample-level scores,
c) processes the descriptor strings into natural language forms, and
d) selects from a coffee's generated descriptor pool for descriptor output.

97. The coffee evaluation system of claim 96, wherein a first evaluative criteria comprise categories that are similar to attributes on a cupping form and provide context for a description but not descriptive detail.

98. The coffee evaluation system of claim 96, wherein the coffee evaluation system includes a scoring engine that sorts and tallies categories and provides sample-level scores.

99. A coffee evaluation method, the coffee evaluation method comprising:
providing a user interface having a tiered structure of attribute descriptors; and
providing an indication of intensity for at least some of the attribute descriptors.

100. The coffee evaluation method of claim 101, wherein the coffee evaluation method includes a scoring engine.

101. The coffee evaluation method of claim 102, wherein the scoring engine assigns values to individual entries of the attribute descriptors.

102. The coffee evaluation method of claim 101, wherein the coffee evaluation method includes a scoring engine that:
a) assigns values to individual entries on the user interface,
b) calculates values for descriptor strings,
c) sorts and tallies category and then sample-level scores,
d) processes the descriptor strings into natural language forms, and
e) selects from a coffee's generated descriptor pool for descriptor output.

103. The coffee evaluation method of claim 102, wherein a first evaluative criteria comprises categories that are similar to attributes on a common cupping form and provide context for a description but not descriptive detail.
